(19) United States
(12) Patent Application Publication
(10) Pub. No.: US 2004/0158329 A1
(43) Pub. Date: Aug. 12, 2004

(54) Hip joint prosthesis with shaft inserted into femur

(75) Inventor: Helmut D. Link, Hamburg (DE)

Correspondence Address: MORRISON & FOERSTER LLP, 1650 TYSONS BOULEVARD, SUITE 300, MCLEAN, VA, 22102, US

(73) Assignee: Waldemar Link (GmbH & Co.), Branford, CT

(21) Appl. No.: 10/361,007

(22) Filed: Feb. 10, 2003

(51) Int. Cl. A61F 2/36

(52) U.S. Cl. 623/23.35; 623/23.36

(57) ABSTRACT

Hip-joint prosthesis with a stem (12) which is to be inserted into the femur and which comprises a part (13) projecting into the trochanteric region (4) of the bone. For the purpose of more intimate contact with the bone, this part (13) is provided with a coating (16) which contains an osteoinductive substance.

The spongy bone tissue in the metaphysis of the femur has a complicated structure of bone trabeculae via which the parts of the bone subjected to compression loads and tensile loads at the femoral neck, the greater trochanter, the lesser trochanter and the diaphysis are connected in a manner transmitting compression and tension. In their totality, they form continuous tension and compression trajectories (Farbatlanten der Medizin [Color Atlas of Medicine], Volume 7: Locomotor apparatus I., published by Thieme Verlag, Stuttgart, 1992). When the stem of a hip-joint prosthesis is inserted, the primary tension trajectories in particular which connect the femoral neck to the opposite intertrochanteric surface area of the bone are for the most part interrupted. When they are then no longer involved in the transmission of forces, they regress. This applies in particular when using prostheses whose prosthesis stem is clamped in the diaphysis and in which the proximal, metaphyseal region of the femur is barely involved in force transmission, especially in its lateral part. Attempts have been made, using what are referred to as tension anchors, to connect the prosthesis stem to the area of the greater trochanter and in this way to involve the latter in the flow of forces. A rod connected to the prosthesis stem was guided through the greater trochanter and provided on the outside with a locking nut so that, upon loading of the hip prosthesis, a tension is exerted on the greater trochanter (U.S. Pat. No. 3,995,323, EP-B-93230, DE-B-1943598). However, it has been found that mechanical tension anchors of this kind quickly come loose as a result of the constant alternating loading and therefore are effective only for a short time. It is also known to design the stem, or a wing projecting laterally from it into the region of the greater trochanter, in such a way that an intimate connection is obtained with the bone substance growing into pores or openings of this wing (GB-A-1030145, FR-A-2356465, EP-A-128036, EP-A-222236, EP-A-95440, EP-B-601223, EP-A-1044665). To promote the connection of the bone with the prosthesis surface, it is also known to make the prosthesis surface osteoconductive. This term denotes surfaces which tolerate adjacent bone growth. These include surfaces made of titanium alloys and coatings which contain calcium phosphate or hydroxyapatite.

The object of the invention is to make available a prosthesis which, used as a hip prosthesis, ensures that the trochanteric region of the metaphysis of the femur is more strongly involved in the flow of forces. The solution according to the invention lies in the features of the claims.

Accordingly, provision is made for at least part of the trochanteric surface of the prosthesis to comprise an osteoinductive substance. Osteoinductive substances, in contrast to osteoconductive substances, are to be understood as substances which stimulate undifferentiated pluripotent stem cells to convert to bone cells (Albrechtsson, Johansson: Osteoinduction, Osteoconduction and Osseointegration; in: Gunzburg Press: The use ob [sic] bone substitutes in spine surgery; Springer. Denissen, H. et al.: Ceramic hydroxyapatite implants for the release of bisphosphonate; in: Bone and Mineral 1994, pages 123-124. Yoshinari, M. et al.: Bone response sto [sic] calcium phosphate-coated and bisphosphonate-immobilized titanium implants; in: Biomaterials 2002, pages 2879-2885. Yoshinari, M. et al.: Immobilization of bisphosphonates on surface-modified titanium; in: Biomaterials 2002, pages 709-715). These substances include bisphosphonates or BMPs (bone morphologic proteins). These are preferred in the use according to the invention.

It is particularly expedient for the substance to be incorporated into a coating which is also intended to be porous on the outside. The coating can be of any desired type. For example, it can be a porous metal layer. Coatings of particular advantage are ones which as such are originally osteoconductive and for example consist of calcium phosphate or hydroxyapatite.

The effect of the invention is that, very quickly after the operation, bone cells develop in immediate proximity to and in connection to the prosthesis surface. The result of this is that relative movements between the bone surface and the bone do not initially cause formation of a gap or intermediate connective tissue layer which makes the later intimate contact more difficult or impossible. The invention is based on the realization that, even in cases where there appeared to be a close connection between the prosthesis surface and the bone substance, there was, in reality, a dividing, albeit microscopically thin intermediate layer present. By virtue of the invention, there is a more rapid accumulation of bone on the trochanteric surface of the prosthesis, and more rapid incorporation of bone into the pores and recesses of said trochanteric surface, so that the trochanteric region of the bone quickly achieves a permanent connection to the prosthesis and, as a result of this, is involved in the transmission of forces.

The measure according to the invention is normally provided only for the trochanteric region of the prosthesis. Although the scope of the invention is not intended to exclude the possibility of osteoinductively modified surfaces being used in other prosthesis regions too, this is not generally desirable since, because of the resulting intimate contact, this can make it difficult to free the prosthesis from the bone in cases of follow-up surgery.

The surface area of the trochanteric prosthesis part containing the osteoinductive substance expediently has pores or undercuts in relation to the lateral direction, so that the bone substance formed as a result of the osteoinduction can not only adhere to the surface but can also anchor onto it with a form fit.

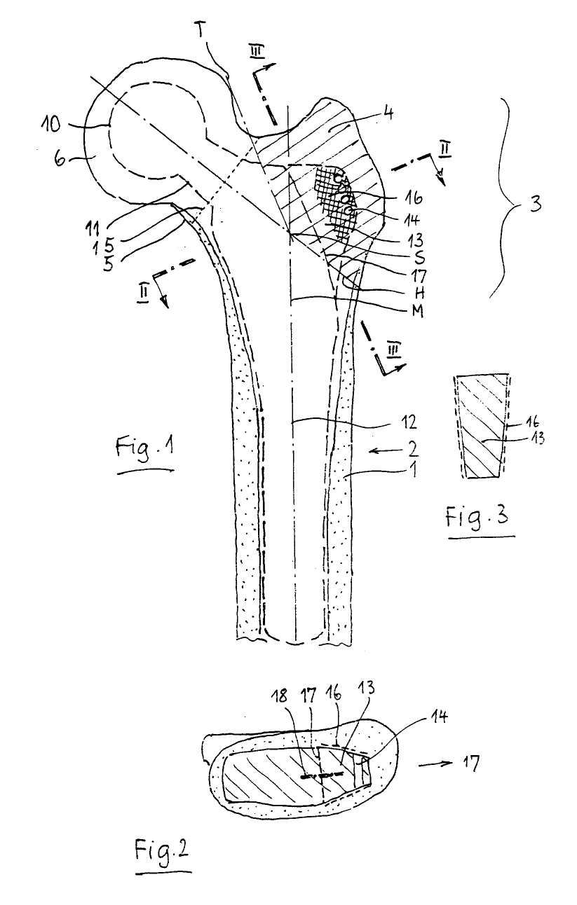

The trochanteric surface of the prosthesis is to be regarded as the surface which, after normal implantation, is intended to lie within the trochanteric region of the femur. The trochanteric part of the bone is the hatched part in FIG. 1. Starting from the point of intersection between the mid-line of the femoral neck and the mid-line of the proximal end of the diaphysis, the trochanteric region lies laterally from the tangent drawn from this point of intersection to the upper edge of the head of the hip, and laterally from that part of the mid-line of the femoral neck continuing this tangent. The trochanteric surface of the prosthesis is that part of its surface which is intended to lie in the trochanteric part of the bone. This surface part can be easily determined on the prosthesis before it is implanted, since one knows how it is to be implanted and which position it will accordingly normally adopt in the bone.

In the middle of the trochanteric part of the bone, the spongy substance is sometimes less dense than it is near the cortex. For this reason, the portions of the trochanteric, osteoinductive surface parts of the prosthesis pointing in the ventral and dorsal directions are preferably located at a certain distance from the mid-plane of the bone. Therefore, the part of the prosthesis forming these surface portions ought not to be too thin in the AP direction. Its thickness, and thus the distance between said surface portions, is expediently over 6 mm, and more advantageously over 9 mm to approximately 15 mm.

The growth of fresh bone cells onto the prosthesis surface can be promoted by a press-fit of the surface portions in question. It is therefore expedient if the surfaces in question, and their mating surfaces, are made wedge-shaped in the direction in which the prosthesis is inserted into the bone, and if the rasp assigned to the prosthesis, and used to shape the receiving area for the prosthesis stem, is provided with slightly smaller cross-sectional dimensions so that, when the prosthesis stem is inserted into the space formed by the rasp, the surface portions in question displace bone substance.

The invention is explained in more detail below with reference to the drawing, in which:

FIG. 1 shows a longitudinal section through the proximal portion of the femur, in the plane containing the longitudinal axis of the handle,

FIG. 2 shows a cross section along the line II-II in FIG. 1, and

FIG. 3 shows a section through the prosthesis along the line III-III in FIG. 1.

The outer contour of the bone is shown by solid lines in FIG. 1. The inner boundary line of the compact cortex 1 is also indicated with solid lines where its thickness is appreciable. This is the case in the area of the diaphysis 2. In the metaphysis 3, the cortex is for the most part so thin that it has only been indicated by a line. The metaphysis 3, and also some of the upper part of the diaphysis, is filled with spongy bone substance (not shown). In the present case, it is only what is known as the trochanteric region 4 related to the greater trochanter that is of interest in this regard, and this is shown by hatching. For the purposes of the present invention, it is defined by the boundary lines H and T. The first of these is the mid-line of the femoral neck 5 and the head 6 of the hip. The second is the tangent to the head 6 of the hip from the point S where the line H and the center axis M of the proximal diaphysis intersect in the plane of the drawing. The trochanteric part of the bone also includes the bone parts situated in front of and behind the plane of the drawing in the area of the hatched surface 4.

The prosthesis to be inserted into the bone is indicated by broken lines in FIG. 1 and by solid lines in FIGS. 2 and 3. It consists of a head 10, a neck 11 and a stem 12, from which a projection 13 protrudes into the trochanteric region 4 of the bone. The projection 13 is provided with bores 14 which form surface areas facing in the ventral direction. These are undercut in relation to the lateral direction 17. Bone substance which grows into these bores grips behind the undercut surfaces and thereby contributes to transmitting tensile forces from the prosthesis to the trochanteric bone region. It will be appreciated that the projection and the undercut surfaces can also be designed in another known way. For example, the projection can be blade-shaped or bow-shaped. Instead of bores, it can have larger apertures or ribs for forming undercut surfaces. Those parts of the prosthesis which in normal circumstances are intended to lie in the trochanteric region 4 after correct implantation are designated as the trochanteric parts or trochanteric surface of the prosthesis. These include in particular the projection 13.

A prosthesis type is shown whose stem 12 is designed and implanted in such a way that it is firmly anchored in the diaphysis 2 of the bone. This type of anchoring leads to great relief of the pressure on the trochanteric bone region. For this reason, the use of the invention is particularly advantageous for this prosthesis type. However, it can also be used in other prosthesis types which are anchored to a large extent in the metaphysis and/or which bear on the resected surface 15 of the bone via a collar (not shown).

The surface of the stem 12 or parts thereof can be designed or equipped in a known manner to promote the connection with the bone. For example, a coating can be provided which is porous and/or consists of calcium phosphate or hydroxyapatite. Such a layer can also be provided wholly or partially in the trochanteric region of the prosthesis. For example, the cross-hatching in FIG. 1 and broken lines in FIGS. 2 and 3 indicate an area 16 which is provided with a coating. This coating, according to the invention, contains an osteoinductive substance.

The osteoinductive coating in the corresponding areas 16 leads to intensive bone growth in direct contact with the prosthesis surface and also in any undercut areas. Therefore, the forces exerted on the prosthesis can be effectively transmitted from its surface to the trochanteric region 4 of the bone. This is therefore involved in the flow of forces, and bone breakdown is prevented.

FIG. 2 illustrates that the projection 13 has a substantial thickness in the anteroposterior direction. Its anterior and posterior surface portions 16 are therefore removed from the middle area in which the spongy bone substance is in many cases depleted, and they are situated in a denser area nearer the cortex. The probability of a good connection between the bone surface and the bone substance is thereby further increased.

FIG. 3 illustrates the cross-sectional shape of the projection 13 in the direction III-III which also corresponds approximately to the direction of insertion. If the shape of the cavity which has been prepared, by means of a rasp, for receiving the prosthesis is slightly smaller than the prosthesis shape, the insertion of this wedge shape into the bone is associated with a displacement of bone substance and thus with an increase of the pressing exerted by the bone substance on the prosthesis surface. This also promotes a rapid and intimate contact of the prosthesis surface with the bone.

In the example illustrated, only the coating 16 of the projection 13 is provided with an osteoinductive substance. The intimate contact thus obtained between the prosthesis surface and the bone can be problematic in the event of a revision because the prosthesis parts in question are difficult to remove from the bone without damaging said bone. According to a special feature of the invention, the projection 13 is therefore designed so that it can be detached from the stem 12. For example, along a connecting joint 17 indicated by broken lines in FIGS. 1 and 2, it can be connected by means of screws 18 or other connecting means to the stem 12, and it can be detached from the latter before the stem is removed from the bone. The projection can then be more easily released from the bone surrounding it and growing onto it.

1. Hip-joint prosthesis with a stem (12) which is to be inserted into the femur and which has a trochanteric surface part intended to lie in the trochanteric region (4), characterized in that at least part of the trochanteric surface of the prosthesis comprises an osteoinductive substance.

2. Hip-joint prosthesis according to claim 1, characterized in that the osteoinductive substance is formed by a coating or is part of a coating.

3. Hip-joint prosthesis according to claim 1 or 2, characterized in that the osteoinductive substance includes a bisphosphonate or a BPM.

4. Hip-joint prosthesis according to claim 1 or 2, characterized in that at least the part of the trochanteric prosthesis surface containing the osteoinductive substance is porous.

5. Hip-joint prosthesis according to claim 1 or 2, characterized in that the osteoinductive substance is applied on surface portions of the trochanteric prosthesis surface which are undercut with respect to the lateral direction (17).

6. Hip-joint prosthesis according to claim 1 or 2, characterized in that the trochanteric prosthesis surface comprises a ventral surface and a dorsal surface which are provided with an osteoinductive coating (16) and are at a distance from one another, in the AP direction, of more than 6 mm, preferably more than 9 mm.

7. Hip-joint prosthesis according to one of claims 1 to 6, characterized in that the trochanteric prosthesis surface is formed at least partially by a projection (13) protruding from the stem (12).

8. Hip-joint prosthesis according to claim 7, characterized in that the projection (13) is wedge-shaped in the direction of implantation, and a rasp assigned to the prosthesis has a smaller volume in the area of this projection.

9. Hip-joint prosthesis according to claim 8, characterized in that the projection (13) can be detached from the stem.
